(19) United States
(12) Patent Application Publication
(10) Pub. No.: US 2026/0357062 A1
(43) Pub. Date: Nov. 12, 2026

(54) THERMOPLASTIC MATERIAL FOR DISPLAY DEVICE, MANUFACTURING METHOD THEREOF, AND DISPLAY DEVICE COMPRISING THE SAME

(71) Applicant: SAMSUNG ELECTRONICS CO., LTD., Suwon-si (KR)

(72) Inventors: Dongwoon KIM, Suwon-si (KR); Seungsan HAN, Suwon-si (KR)

(73) Assignee: SAMSUNG ELECTRONICS CO., LTD., Suwon-si (KR)

(21) Appl. No.: 19/720,364

(22) Filed: Jun. 25, 2026

Related U.S. Application Data
(63) Continuation of application No. PCT/KR2026/001554, filed on Jan. 27, 2026.

(30) Foreign Application Priority Data
May 2, 2025 (KR) 10-2025-0058546

(51) Int. Cl. C08L 69/00; C08L 25/06; C08L 27/00; C08L 55/00; C12P 7/6409; H05K 5/03

(52) U.S. Cl. CPC C08L 69/00; C08L 25/06; C08L 27/00; C08L 55/00; C12P 7/6409; H05K 5/03

(57) ABSTRACT

The present disclosure relates to a thermoplastic material for display devices, a method for manufacturing the same, and a display device comprising the same. By utilizing microalgae as a raw material for thermoplastic resin production, carbon emissions generated during the plastic material manufacturing process may be effectively reduced.

### CROSS REFERENCE TO RELATED APPLICATIONS

This application is a continuation of International Application No. PCT/KR2026/001554 filed Jan. 27, 2026, which claims priority from Korean Patent Application No. 10-2025-0058546 filed May 2, 2025, the disclosure of which is incorporated herein by reference in its entirety.

### BACKGROUND

### 1. Field

The disclosure relates to a thermoplastic material for a display device, a method for manufacturing the same, and a display device including the same.

### 2. Description of Related Art

Plastics are widely used in various industrial fields due to their excellent mechanical properties, thermal properties, dimensional stability, chemical resistance, and weather resistance. In particular, acrylonitrile butadiene styrene copolymer (ABS) resins, epoxy resins, polyacetal resins, and thermoplastic polyurethane (TPU) resins each exhibit unique physical properties and are applied in various fields including electronic devices such as display devices and printed circuit boards.

Conventionally, plastics are manufactured through a series of processes including extraction, refining, and processing of raw materials obtained from fossil fuels such as petroleum and natural gas. During such processes, a significant amount of carbon dioxide (CO₂) is generated using fossil fuel-based energy, and the carbon dioxide thus generated is identified as a major cause of accelerating global warming. Countries and companies around the world are seeking various measures to reduce such greenhouse gas emissions, but the carbon emission problem occurring in the plastic manufacturing process itself remains difficult to solve.

Conventional plastic manufacturing processes have been performed by fractionally distilling crude oil drilled from underground to extract naphtha, synthesizing monomers using the naphtha, and then producing plastics through polymerization reactions.

However, the above-described conventional plastic manufacturing processes emit carbon throughout the entire process, and from a Life Cycle Assessment (LCA) perspective, may cause various environmental problems such as exacerbating climate warming and environmental pollution.

### SUMMARY

An embodiment of the present disclosure provides a thermoplastic material for a display device formed from a thermoplastic resin derived from microalgae.

An embodiment of the present disclosure provides a method for manufacturing a thermoplastic material for a display device, the method including a carbon fixation step having a negative carbon emission factor, wherein a carbon reduction rate compared to petroleum raw materials in the entire plastic material manufacturing process is 3.65% or more.

An embodiment of the present disclosure provides a display device comprising a plastic material formed from a thermoplastic resin derived from microalgae.

A thermoplastic material for display devices according to an embodiment of the present disclosure may include a thermoplastic resin obtained from bio-naphtha derived from microalgae, wherein the thermoplastic resin may include at least one selected from the group consisting of polycarbonate-based resins, rubber-modified vinyl copolymer resins, styrene-based resins, and polyolefin-based resins.

A method for manufacturing a thermoplastic material for display devices according to an embodiment of the present disclosure may include: cultivating microalgae to fix intracellular carbon in the microalgae; extracting microalgal oil from the microalgae; forming bio-naphtha from the microalgal oil; producing a thermoplastic resin from the bio-naphtha.

A display device according to an embodiment of the present disclosure may include: a display panel configured to display images; a chassis configured to cover at least a portion of side surfaces and a portion of a front surface of the display panel; and a rear cover configured to cover a rear surface of the display panel, wherein the chassis and the rear cover may include a thermoplastic material, wherein the thermoplastic material may include a thermoplastic resin obtained from bio-naphtha derived from microalgae, and wherein the thermoplastic resin may include at least one selected from the group consisting of polycarbonate-based resins, rubber-modified vinyl copolymer resins, styrene-based resins, and polyolefin-based resins.

According to the present disclosure, it is possible to provide the thermoplastic material for a display device formed from a thermoplastic resin derived from microalgae.

Further, by manufacturing the thermoplastic material using microalgae as a raw material, it is possible to provide the method for manufacturing a plastic material in which a negative carbon emission effect is achieved by carbon fixation by cultivation of microalgae, so that carbon emissions in the process of manufacturing the thermoplastic material may be reduced by 3.65% or more.

Furthermore, in the display device according to the present disclosure, the thermoplastic material constituting the device may include a thermoplastic resin derived from microalgae, carbon emissions may be reduced and the environmental load may be decreased according to the LCA approach.

### BRIEF DESCRIPTION OF DRAWINGS

FIG. 1 schematically illustrates a method of manufacturing a thermoplastic material according to an embodiment of the present disclosure.

FIG. 2 illustrates a perspective view of a display device according to an embodiment of the present disclosure.

FIG. 3 illustrates a rear view of the display device according to an embodiment of the present disclosure.

FIG. 4 illustrates an exemplary view of components for a display device according to an embodiment of the present disclosure.

### DETAILED DESCRIPTION

Various embodiments and terms in this document are not intended to limit the technical features described in this document to specific embodiments, and should be understood to include various modifications, equivalents, or substitutes of the embodiments.

In connection with the description of the drawings, like reference numbers may be used for like or related components.

The singular form of a noun corresponding to an item may include one item or a plurality of items, unless the relevant context clearly dictates otherwise.

In this document, each of phrases such as “A or B,” “at least one of A and B,” “at least one of A or B,” “A, B or C,” “at least one of A, B and C,” and “at least one of A, B, or C” may include any one of the items listed in a phrase, or all possible combinations thereof.

The term “and/or” includes any combination of a plurality of related components or any one of a plurality of related components.

Terms such as “first,” “second,” “primary,” and “secondary” may simply be used to distinguish a given component from other corresponding components, and do not limit the corresponding components in any other aspect (e.g., importance or order).

When any (e.g., a first) component is referred to as being “coupled” or “connected” to another (e.g., a second) component with or without the terms “functionally” or “communicatively,” this means that the any component may be connected to the other component directly (e.g., by a wire), wirelessly, or through a third component.

The terms “includes,” “has,” and the like are intended to indicate that there are features, numbers, steps, operations, components, parts, or combinations thereof described in this document, and do not exclude the presence or addition of one or more other features, numbers, steps, operations, components, parts, or combinations thereof.

When any component is referred to as being “connected,” “coupled,” “supported” or “in contact” with another component, this includes a case in which the components are indirectly connected, coupled, supported, or in contact with each other through a third component as well as directly connected, coupled, supported, or in contact with each other.

When any component is referred to as being located “on” another component, this includes not only a case in which any component is in contact with another component but also a case in which another component is present between the two components. As used herein, the term ‘parts by weight’ may be interpreted as weight percent (wt %).

### Thermoplastic Material for Display Devices

A thermoplastic material for a display device according to an embodiment of the present disclosure may be formed from a thermoplastic resin derived from microalgae.

Microalgae may be phytoplankton that inhabit freshwater or marine environments and absorb carbon dioxide (CO₂) through photosynthesis.

The microalgae may include at least one selected from the group consisting of Chlorella, Euglena, Schizochytrium, Nannochloropsis, Isochrysis, Haematococcus, Dunaliella, Crypthecodinium, Tetraselmis, Neochloropsis, Picochlorum, Botryococcus, Coccolithophores, and Cyanobacteria.

According to an embodiment of the present disclosure, the thermoplastic resin derived from microalgae may be obtained from bio-naphtha derived from microalgae.

The thermoplastic material may include 10 to 100 parts by weight of the thermoplastic resin based on 100 parts by weight of the thermoplastic material. When the thermoplastic resin is present in an amount less than 10 parts by weight, the content of the thermoplastic resin in the thermoplastic material may become too low, making it difficult to secure the target physical properties. When the amount of thermoplastic resin is 100 parts by weight or less, an increase in the viscosity of the thermoplastic material may be suppressed, thereby ensuring moldability. Furthermore, when additional materials such as reinforcing agents and flame retardants other than the resin are additionally included, it becomes easier to secure the target physical properties.

The thermoplastic resin may be described in more detail as follows.

The thermoplastic resin may include at least one selected from the group consisting of polycarbonate-based resin, rubber-modified vinyl copolymer resin, styrene-based resin, and polyolefin-based resin.

The polycarbonate-based resin may have excellent mechanical properties such as impact resistance, tensile strength, and rigidity, thereby improving the overall physical properties of the thermoplastic material. The polycarbonate-based resin may include at least one selected from the group consisting of polycarbonate (PC), polycarbonate/acrylonitrile-butadiene-styrene (ABS) blend resin, polycarbonate/polyester blend resin, polycarbonate/polyolefin blend resin, and polycarbonate copolymer.

The rubber-modified vinyl copolymer resin may improve the processability and impact resistance of the thermoplastic material, thereby preventing cracking or breakage of the final product. The rubber-modified vinyl copolymer resin may include at least one selected from the group consisting of acrylonitrile-butadiene-styrene (ABS), methyl methacrylate-butadiene-styrene (MBS), acrylonitrile-styrene-acrylate (ASA), acrylonitrile-EPDM-styrene copolymer (AES), acrylonitrile-acrylate-styrene (AAS), and other rubber-modified vinyl resins. Here, the rubber-modified vinyl resin may include a rubber-modified aromatic vinyl graft copolymer in which aromatic vinyl monomers and alkyl methacrylate monomers are grafted onto a rubber polymer. Additionally, the rubber-modified vinyl copolymer resin may include at least one selected from the group consisting of modified SAN in which methyl methacrylate (MMA) is grafted to styrene-acrylonitrile (SAN) and acrylonitrile-butadiene-styrene (ABS). For example, when ABS is used as the rubber-modified aromatic vinyl graft copolymer, deterioration of fluidity and impact properties may be prevented, making it easy to secure mechanical properties. Additionally, the ABS applied in the present disclosure may be recycled ABS.

The styrene-based resin may have excellent fluidity and moldability, enabling precise molding of parts with complex shapes, and may improve the appearance quality of the final product due to excellent surface gloss and dimensional stability. The styrene-based resin may include at least one selected from the group consisting of polystyrene (PS), high impact polystyrene (HIPS), styrene-acrylonitrile (SAN), styrene-maleic anhydride (SMA), styrene-butadiene (SB), and styrene block copolymer (SBC).

The polyolefin-based resin may have excellent chemical resistance, moisture resistance, and electrical insulation properties, thereby securing the durability and safety of the final product. The polyolefin-based resin may include at least one selected from the group consisting of polypropylene (PP), high density polyethylene (HDPE), low density polyethylene (LDPE), linear low density polyethylene (LLDPE), ethylene-propylene copolymer (EP), ethylene-butene copolymer (EB), and ethylene-based elastomer.

In the thermoplastic resin according to an embodiment of the present disclosure, at least one of polycarbonate, rubber-modified vinyl copolymer resin, styrene-based resin, and polyolefin-based resin may be obtained from bio-naphtha derived from microalgae.

In other words, by manufacturing plastic materials using microalgae as raw materials, carbon emissions generated during the manufacturing process may be effectively reduced compared to conventional fossil fuel-based plastic materials.

The thermoplastic material according to an embodiment of the present disclosure may further include at least one of phosphorus-based flame retardants, inorganic reinforcing materials, and additives as needed.

The phosphorus-based flame retardant may be a flame retardant containing phosphorus (P) that forms a char layer on the surface of the thermoplastic material, and such a char layer may act as a physical barrier for the thermoplastic material. In other words, the char layer formed by the flame retardant may block heat transfer and gas diffusion to the thermoplastic material at high temperatures. However, in the case of thermoplastic material to which low molecular weight phosphorus-based flame retardants are applied, the flame retardant effect may be reduced due to deterioration of durability, hygroscopicity, migration, adhesion, water resistance, and the like. Therefore, in an embodiment of the present disclosure, high molecular weight phosphorus-based flame retardants having excellent flame retardant effects and large molecular weights may be applied. The phosphorus-based flame retardant may include at least one selected from the group consisting of phosphate compounds, phosphonate compounds, phosphinate compounds, phosphine oxide compounds, and phosphazene compounds. Additionally, even if not explicitly described herein, the phosphorus-based flame retardant may be one adopted by those skilled in the art as needed. The phosphorus-based flame retardant may be included in an amount of 5 to 30 parts by weight based on 100 parts by weight of the thermoplastic material. When the phosphorus-based flame retardant is added in an amount less than 5 parts by weight, flame retardancy and fluidity may be reduced, or the appearance of the plastic material may be impaired, such as the formation of gate marks and/or flow marks. When the amount of phosphorus-based flame retardant exceeds 30 parts by weight, impact properties and dimensional stability may be deteriorated or a poor appearance may result.

The thermoplastic material according to an embodiment of the present disclosure may be applied to chassis, covers, stands, or the like of display devices. For such display devices, it is necessary to secure a low coefficient of thermal expansion according to temperature to ensure the components can be assembled. Therefore, the thermoplastic material according to an embodiment of the present disclosure may include inorganic reinforcing materials to prevent shrinkage and expansion according to temperature.

The inorganic reinforcing material may include at least one selected from the group consisting of talc, mica, glass fiber, wollastonite, calcium carbonate, and kaolin as inorganic fillers. The inorganic reinforcing material may be included in an amount of 10 to 50 parts by weight based on 100 parts by weight of the thermoplastic material. When the inorganic reinforcing material is present in an amount less than 10 parts by weight, flame retardancy may be deteriorated or dimensional stability may be difficult to achieve. When the amount of the inorganic reinforcing material exceeds 50 parts by weight, impact strength may be impaired or dimensional stability may be difficult to achieve.

The thermoplastic material according to an embodiment of the present disclosure may optionally include 10 to 30 parts by weight of additives based on 100 parts by weight of the thermoplastic material. The additives may include one or more of lubricants, antioxidants, flame retardant auxiliaries, and heat stabilizers. Additionally, even if not explicitly described in this specification, the additive may be one adopted by those skilled in the art as needed.

The thermoplastic material according to an embodiment of the present disclosure may have a notched Izod impact strength of 3 kgf-cm/cm or more for a ⅛″ thick specimen measured according to ASTM D256. Through this, it may be determined whether the thermoplastic material of the present disclosure may secure high strength.

The thermoplastic material according to an embodiment of the present disclosure may have a melt-flow index of 10 to 60 g/10 min measured under conditions of 260 °C and 2.15 kgf load according to ASTM D1238. By measuring the melt flow index of the thermoplastic material according to an embodiment of the present disclosure, it may be determined whether the thermoplastic material of the present disclosure may secure fluidity, and thereby whether injection molding can be easily carried out may be known.

The thermoplastic material according to an embodiment of the present disclosure may have a flame retardancy of V-0 or higher for a 1.2 mm thick specimen measured according to UL-94 standards. By measuring the flame retardancy with a specimen of the thermoplastic material according to an embodiment of the present disclosure, it may be determined whether the thermoplastic material of the present disclosure may secure flame retardancy.

The thermoplastic material according to an embodiment of the present disclosure may have a linear expansion coefficient of 20 to 85 mm/° °C measured for a 6.4 mm flexural specimen at 90 °C according to ASTM D696. The temperature may be raised from 0 °C to 90 °C at a rate of 5 °C/min, and the specimen may refer to an injection molded specimen. By measuring the linear expansion coefficient with a specimen of the thermoplastic material according to an embodiment of the present disclosure, it may be determined whether the thermoplastic material of the present disclosure may secure dimensional stability.

The thermoplastic material according to an embodiment of the present disclosure may have a heat deflection temperature of 75 °C or higher measured for a 6.4 mm thick specimen under a load of 18.56 kgf/cm² according to the evaluation method specified in ASTM D648. The heat deflection temperature may be the temperature at which deformation appears, and it may be determined whether thermal properties may be secured.

The thermoplastic material formed from thermoplastic resin derived from microalgae according to an embodiment of the present disclosure may exhibit physical properties such as mechanical strength, durability, and heat resistance that are equal to or superior to conventional petroleum-based plastic materials. Additionally, the thermoplastic material according to an embodiment of the present disclosure may have a significantly reduced carbon footprint compared to conventional fossil fuel-based plastic materials by utilizing microalgae as raw materials.

In other words, unlike conventional fossil fuel-based plastic materials made from petroleum or natural gas, the thermoplastic material according to the present disclosure may have the effect of reducing carbon emissions generated during the thermoplastic production process by using microalgae as raw materials.

The thermoplastic material according to an embodiment of the present disclosure may be in the form of one of pellets, sheets, films, powder, tubes, bars, preforms, and profiles.

The thermoplastic material according to an embodiment of the present disclosure may be applied to components, such as covers and stands of display devices. Here, the display device may be implemented in various forms such as televisions (TVs), monitors, remote controls, portable multimedia devices, portable communication devices, or the like, as long as it is a device that visually displays an image.

Additionally, components formed by processing the thermoplastic material according to an embodiment of the present disclosure may have a thickness of 0.7 mm to 30.0 mm.

Hereinafter, a method of manufacturing a thermoplastic material for a display device will be described with reference to the drawings.

### Manufacturing Method of Thermoplastic Material for Display Devices

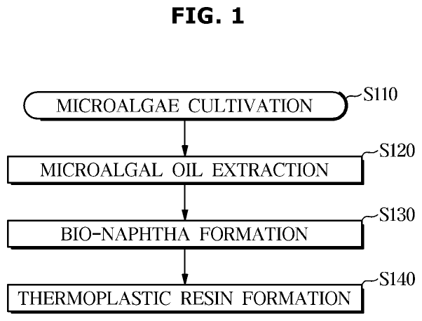

FIG. 1 schematically illustrates a method of manufacturing a thermoplastic material for display devices according to an embodiment of the present disclosure.

As shown in FIG. 1, a method of manufacturing the thermoplastic material according to an embodiment of the present disclosure may include microalgae cultivation (S110), microalgal oil extraction (S120), bio-naphtha formation (S130), and thermoplastic resin formation (S140).

The method of manufacturing a thermoplastic material according to an embodiment of the present disclosure may use microalgae as raw materials to manufacture a thermoplastic material for display devices.

In more detail, the microalgae used as raw materials may be phytoplankton found in freshwater or marine environments, and may include at least one selected from the group consisting of Chlorella, Euglena, Schizochytrium, Nannochloropsis, Isochrysis, Haematococcus, Dunaliella, Crypthecodinium, Tetraselmis, Neochloropsis, Picochlorum, Botryococcus, Coccolithophores, and Cyanobacteria.

In the microalgae cultivation (S110), microalgae may be cultivated to fix carbon within the cells of the microalgae.

In the microalgae cultivation (S110), a reaction occurs in which the microalgae absorb carbon dioxide (CO₂) from the atmosphere through photosynthesis and fix the carbon dioxide in the form of organic matter, such as carbohydrates, lipids, and proteins within the microalgae cells. That is, atmospheric carbon dioxide may be reduced through such a process, and as a result of evaluation through the LCA approach, a negative carbon emission factor may be exhibited.

In this time, the microalgae cultivation (S110) may be performed in an open pond method. Accordingly, the use of fuel and energy accompanying microalgae cultivation may be minimized to reduce carbon emissions and manufacturing costs.

Next, in the microalgal oil extraction (S120), microalgal oil may be extracted from the microalgae in which intracellular carbon is fixed.

In the microalgal oil extraction (S120), microalgal oil may be extracted by using at least one method selected from the group consisting of solvent extraction, microwave utilization, hydrothermal treatment, enzymatic treatment, and pressing methods.

The microalgal oil may include fatty acids, which may include 60% to 90% saturated fatty acids and 10% to 40% unsaturated fatty acids.

Additionally, the fatty acids may be in the form of free fatty acids at 90% or more.

The microalgal oil may contain fixed carbon due to the aforementioned microalgae cultivation (S110), and the carbon fixation amount in the microalgal oil may be 2.57 kgCO₂eq or more. This may be based on the results derived by setting the carbon content per 1 kg of microalgal oil to 70% and converting the corresponding carbon content entirely to carbon dioxide. The calculation expression is as follows:

Carbon fixation amount in microalgal oil=Mass ratio of carbon in microalgal oil (wt %)×(Mass of carbon dioxide molecule/Mass of carbon atom)

At this time, as a result of elemental analysis of the microalgal oil sample, it was identified that approximately 72.02 wt % of carbon was contained in the microalgal oil, and based on the results, the mass ratio of carbon in the microalgal oil was set to 70 wt % in the present disclosure.

Meanwhile, bio-naphtha may be formed (S130) using the extracted microalgal oil.

In the bio-naphtha formation (S130), at least one process selected from hydrotreating processes and co-processing processes may be performed to form bio-naphtha.

The hydrotreating process may be a process in which microalgal oil reacts with hydrogen in a high-temperature, high-pressure hydrogen atmosphere to remove oxygen in free fatty acids in the form of water (H₂O) or carbon dioxide (CO₂), and convert the remaining carbon into saturated hydrocarbons that are precursors of bio-naphtha.

The co-processing process may be a process in which microalgal oil is blended with conventional petroleum refining processes and processed together. Such a process may reduce carbon emissions of the entire production process of plastic materials and improve production efficiency by using fossil fuels and microalgae raw materials simultaneously.

At this time, the bio-naphtha may have a carbon number of C5 to C12.

When the carbon number of bio-naphtha is less than C5, volatility may increase excessively, making storage or processing difficult, and when it exceeds C12, there may be problems such as increased viscosity or decreased vapor pressure, resulting in poor reactivity.

Next, thermoplastic resin may be formed (S140) utilizing the bio-naphtha.

In the thermoplastic resin formation (S140), bio-naphtha may be converted into monomers by refining, reforming, and cracking, and the resulting monomers may be selectively polymerized to form thermoplastic resin.

The thermoplastic resin thus formed may be one or more of a polycarbonate-based resin, a rubber-modified vinyl copolymer resin, a styrene-based resin, and a polyolefin-based resin.

Once the thermoplastic resin is formed by process described above, phosphorus-based flame retardants, inorganic reinforcing materials, additives, and the like may be included to secure the target physical properties required for the final product.

In other words, the thermoplastic material according to an embodiment of the present disclosure may include thermoplastic resin and may optionally include additional materials as needed. The thermoplastic material may be in the form of one of pellets, sheets, films, powder, tubes, bars, preforms, and profiles.

As such, the method of manufacturing thermoplastic material according to an embodiment of the present disclosure may effectively reduce carbon emissions compared to conventional methods of manufacturing thermoplastic material by cultivating and processing microalgae to extract microalgal oil and converting the micoalgal oil into bio-naphtha through a hydrotreating process and/or a co-processing process.

Based on a comparison of carbon emissions with conventional methods, the method of manufacturing thermoplastic material according to an embodiment of the present disclosure may control the carbon reduction rate represented by the following expression (1) to 3.65% or more.

{(petroleum-based carbon emission factor)−(microalgae-based carbon emission factor)}/(petroleum-based carbon emission factor)×100(%) Expression (1):

(In expression (1), the petroleum-based carbon emission factor denotes the carbon emission factor when manufacturing thermoplastic material from petroleum raw materials, and the microalgae-based carbon emission factor denotes the carbon emission factor when manufacturing thermoplastic material from microalgae raw materials.)

In the method of manufacturing thermoplastic material according to the present disclosure, the carbon reduction rate may be controlled to 3.65% or more in various ways. For example, in microalgae cultivation (S110), by optimizing the power consumption for driving paddles in open pond systems, the carbon emission factor may be reduced, and accordingly, the carbon reduction rate of expression (1) may be increased.

In other words, the method of manufacturing thermoplastic material according to an embodiment of the present disclosure may include a step of fixing atmospheric carbon dioxide to microalgae cells through photosynthesis during the microalgae cultivation process, thereby calculating the carbon dioxide absorption and fixation effect by photosynthesis of microalgae as negative carbon emissions from an LCA perspective, effectively reducing carbon emissions generated during the plastic production process.

### Display Device

A display device according to an embodiment of the present disclosure may include: a display panel configured to display an image; a chassis configured to cover at least a portion of side surfaces and a portion of a front surface of the display panel; and a rear cover configured to cover a rear surface of the display panel.

In this time, the chassis and the rear cover may be formed including the thermoplastic material according to an embodiment of the present disclosure.

The display device and its components will be described in more detail with reference to the drawings.

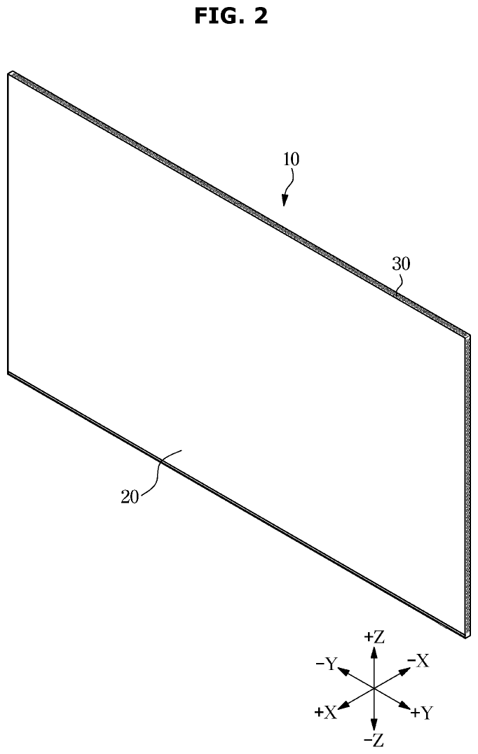

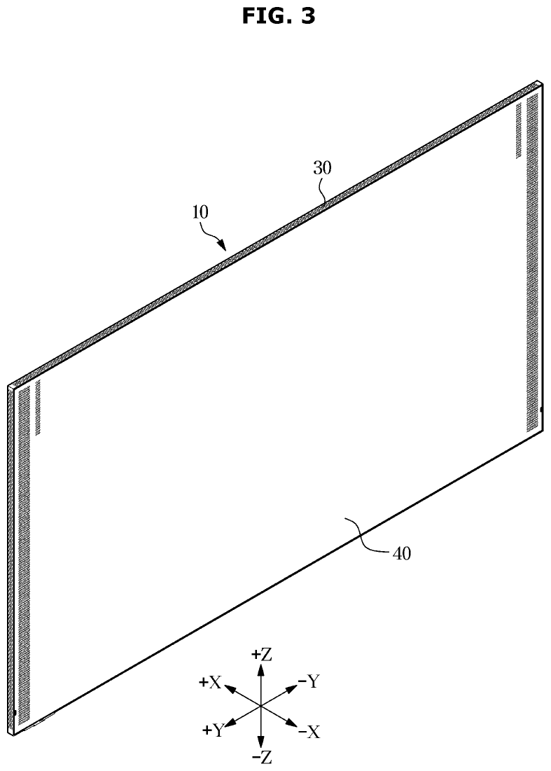

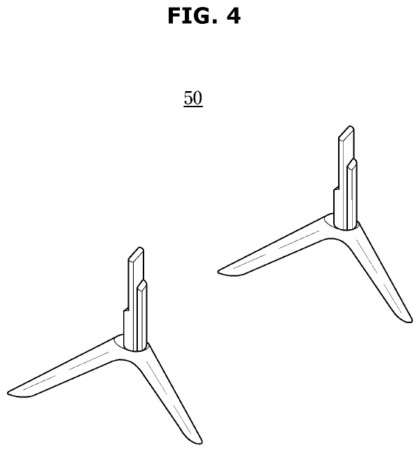

FIG. 2 illustrates a perspective view of a display device according to an embodiment of the present disclosure, FIG. 3 illustrates a rear view of the display device according to an embodiment of the present disclosure, and FIG. 4 illustrates an exemplary view of components for the display device according to an embodiment of the present disclosure.

Referring to FIGS. 2 and 3, the display device 10 may be a device capable of processing image signals received from an external source and visually displaying the processed images. Hereinafter, a case where the display device 10 is a television (TV) is illustrated, but the present disclosure is not limited thereto. For example, the display device 10 may be implemented in various forms, such as monitors, portable multimedia devices, portable communication devices, and the like, and the display device 10 is not limited in form as long as it is a device that visually displays images.

The display device 10 may receive content data including video data and audio data from various content sources, and may output video and audio corresponding to the video data and audio data. For example, the display device 10 may receive content data through a broadcast receiving antenna or a wired cable, may receive content data from a content playback device, or may receive content data from a content provider's content providing server.

The display device 10 may include a display panel 20 configured to display images. The display panel 20 may display images toward the front (X).

The display device 10 may include a front chassis 30 configured to cover at least a portion of side surfaces and a portion of a front surface of the display panel 20.

According to an embodiment, the display device 10 may include a non-emissive panel. For example, the display device 10 may include the display panel 20 and a backlight unit configured to provide light to the display panel 20. For example, the display panel 20 may be a liquid crystal panel.

According to an embodiment, the display device 10 may include a self-emissive panel. In this case, the display panel 20 may be configured to emit light by itself and may not include a backlight unit that provides light to the display panel 20. For example, the display panel 20 may be an organic light emitting diode (OLED) panel. As another example, the display panel 20 may be a micro LED panel in which light emitting diodes are less than 100 micrometers (μm) in size.

The front chassis 30 may be configured to cover side surfaces of the display panel 20. The front chassis 30 may be configured to cover a portion of the front surface of the display panel 20. Specifically, the front chassis 30 may be configured to cover a border of the front surface of the display panel 20. The front portion of the front chassis 30 exposed to the front of the display device 10 may be referred to as a bezel.

The front chassis 30 may be formed to extend along the perimeter of the display panel 20. The front chassis 30 may be configured in a substantially rectangular ring shape. The front chassis 30 may protect the display panel 20 by covering side surfaces and a portion of the front surface of the display panel 20, and may form the side appearance of the display device 10. Additionally, the front chassis 30 may form a part of the front appearance of the display device 10.

As shown in FIG. 3, the display device 10 may include a rear cover 40 configured to cover the rear surface of the display panel 20.

The rear cover 40 may be configured to cover the rear surface of the display panel 20. The rear cover 40 may be disposed behind the display panel 20. The rear cover 40 may be configured with a size and shape corresponding to the display panel 20.

The front chassis 30 and the rear cover 40 may be formed including thermoplastic materials.

The thermoplastic material may include thermoplastic resin obtained from bio-naphtha derived from microalgae.

The thermoplastic resin may include at least one selected from the group consisting of polycarbonate-based resin, rubber-modified vinyl copolymer resin, styrene-based resin, and polyolefin-based resin.

According to an embodiment, the display device 10 may further include a stand 50 for supporting the display device 10.

Referring to FIG. 4, the stand 50 may be configured as a pair, but is not limited thereto, and it should be appreciated that the display device may be supported by a single stand.

One side of the stand 50 may be coupled to the rear cover 40 of the display device 10 to maintain the display device 10 at a certain height from a plane. The stand 50 may have strength capable of stably supporting the weight of the display device 10, and may be designed with a foldable structure when necessary to improve portability of the display device 10. The stand 50 may be hinge-coupled to the rear cover 40 of the display device 10 to adjust the tilt angle of the display device 10, and may position the display device 10 to suit the user's viewing angle. Additionally, the stand 50 may provide a function for rotating the display device 10 in a direction desired by the user.

With such a configuration, the display device according to the present disclosure may include a display panel, a chassis, and a rear cover, wherein the chassis and the rear cover may include a thermoplastic material formed from thermoplastic resin derived from microalgae, thereby contributing to carbon footprint reduction and minimizing environmental impact.

Although the drawings of the present disclosure illustrate a chassis, a cover, and a stand for a display device, the thermoplastic material may be applied as a material constituting various other components for a display device.

However, the drawings of the display device according to an embodiment of the present disclosure are for describing the technical ideas of the present disclosure as described above, and do not limit the scope of rights of the present disclosure.

Hereinafter, examples are described to facilitate understanding of the present disclosure. However, the following description is only an example regarding the content and effects of the present disclosure, and the scope and effects of the present disclosure are not necessarily limited thereto.

### Examples

According to an embodiment of the present disclosure, microalgae were cultivated in an open pond cultivation method, microalgal oil was extracted, bio-naphtha was formed by a hydrotreating process method, and then the bio-naphtha was converted into monomers by refining, reforming, and cracking, and the resulting monomers were polymerized to produce polycarbonate-based resin, rubber-modified vinyl copolymer resin, styrene-based resin, and polyolefin-based resin, respectively.

The thermoplastic resins produced above were used to produce thermoplastic materials with the compositions shown in Table 1 below.

The units in Table 1 below are parts by weight based on 100 parts by weight of the thermoplastic material. In addition, when the total amount of the components for each thermoplastic material is less than 100 parts by weight, the remaining portion may be additives as described in the present specification.

TABLE 1

| | Thermoplastic resin | | | | | |
|---|---|---|---|---|---|---|
| | | Rubber- | | | | |
| | | modified | | | Phosphorus- | |
| | Polycarbonate- | vinyl | Polyolefin- | Styrene- | based | Inorganic |
| | based | copolymer | based | based | flame | reinforcing |
| Classification | resin | resin | resin | resin | retardant | material |
| Thermoplastic | 50~90 | — | — | — | 5~30 | — |
| material A | | | | | | |
| Thermoplastic | 40~80 | — | — | — | 5~30 | 10~20 |
| material B | | | | | | |
| Thermoplastic | 10~70 | 10~30 | — | — | 5~30 | — |
| material C | | | | | | |
| Thermoplastic | 10~60 | 10~25 | — | — | 5~30 | 10~40 |
| material D | | | | | | |
| Thermoplastic | — | 50~90 | — | — | 5~30 | — |
| material E | | | | | | |
| Thermoplastic | — | — | 10~60 | — | 5~30 | 10~50 |
| material F | | | | | | |
| Thermoplastic | — | — | — | 80~100 | — | — |
| material G | | | | | | |

The thermoplastic materials A to G in Table 1 above were designated as Examples A to G, respectively, and Comparative Examples A to G were prepared by carrying out the same procedures as the Examples, except that materials A′ to G′ were prepared using conventional petroleum-based thermoplastic resins instead of the plastic materials A to G in the Examples. Each of the Examples and the Comparative Examples was prepared in 1 kg quantities, and the carbon emissions were subsequently measured.

For each of the Examples and Comparative Examples, the energy and resource consumption occurring in each process and the resulting carbon emissions were calculated using the LCA approach and shown in Table 2 below.

TABLE 2

| | | Carbon emission factor by process | | | | | | |
|---|---|---|---|---|---|---|---|---|
| | | (kgCO₂eq) | | | | | | |
| | | Raw material processing and | | | | | | |
| | | naphtha production | | | | | | |
| | | Micro | | | | | | |
| | | algae | | | | | Total | Carbon |
| | | cultivation | | | | Thermoplastic | carbon | reduction |
| | Thermoplastic | and | Carbon | Petroleum | Naphtha | material | emission | rate |
| Classification | material | extraction | fixation | drilling | production | manufacturing | factor | (%) |
| Comparative | Thermoplastic | — | — | 0.12 | 0.57 | 4.51 | 5.20 | — |
| Example 1 | material A′ | | | | | | | |
| Example 1 | Thermoplastic | 1.50~2.50 | −2.57 | — | 0.57 | 4.51 | 4.01~5.01 | 3.65~22.88 |
| | material A | | | | | | | |
| Comparative | Thermoplastic | — | — | 0.12 | 0.57 | 4.31 | 5.00 | — |
| Example 2 | material B′ | | | | | | | |
| Example 2 | Thermoplastic | 1.50~2.50 | −2.57 | — | 0.57 | 4.31 | 3.81~4.81 | 3.80~23.80 |
| | material B | | | | | | | |
| Comparative | Thermoplastic | — | — | 0.12 | 0.57 | 4.21 | 4.90 | — |
| Example 3 | material C′ | | | | | | | |
| Example 3 | Thermoplastic | 1.50~2.50 | −2.57 | — | 0.57 | 4.21 | 3.71~4.71 | 3.88~24.29 |
| | material C | | | | | | | |
| Comparative | Thermoplastic | — | — | 0.12 | 0.57 | 3.31 | 4.00 | — |
| Example 4 | material D′ | | | | | | | |
| Example 4 | Thermoplastic | 1.50~2.50 | −2.57 | — | 0.57 | 3.31 | 2.81~3.81 | 4.75~29.75 |
| | material D | | | | | | | |
| Comparative | Thermoplastic | — | — | 0.12 | 0.57 | 2.81 | 3.50 | — |
| Example 5 | material E′ | | | | | | | |
| Example 5 | Thermoplastic | 1.50~2.50 | −2.57 | — | 0.57 | 2.81 | 2.31~3.31 | 5.43~34.00 |
| | material E | | | | | | | |
| Comparative | Thermoplastic | — | — | 0.12 | 0.57 | 1.11 | 1.80 | — |
| Example 6 | material F′ | | | | | | | |
| Example 6 | Thermoplastic | 1.50~2.50 | −2.57 | — | 0.57 | 1.11 | 0.61~1.61 | 10.56~66.11 |
| | material F | | | | | | | |
| Comparative | Thermoplastic | — | — | 0.12 | 0.57 | 2.11 | 2.80 | — |
| Example 7 | material G′ | | | | | | | |
| Example 7 | Thermoplastic | 1.50~2.50 | −2.57 | — | 0.57 | 2.11 | 1.61~2.61 | 6.79~42.50 |
| | material G | | | | | | | |

In Table 2 above, the carbon emission factor by process means the amount of greenhouse gases generated when each process is performed once.

The total carbon emission factor represents the net amount of greenhouse gases generated to produce 1 kg of thermoplastic material from each raw material and is the sum of all carbon emission factors for each process.

The carbon reduction rate is an indicator for comparing the carbon emission factors of the Comparative Examples and the Examples when manufacturing the same plastic material, and the carbon reduction rate for each thermoplastic material may be calculated as follows:

Carbon reduction rate for each thermoplastic material={(Total carbon emission factor of Comparative Example)−(Total carbon emission factor of Example)}/(Total carbon emission factor of Comparative Example)×100(%)

As shown in Table 2, the Comparative Examples using petroleum as a raw material do not show carbon emission factor reduction effects due to carbon fixation.

On the other hand, when manufacturing each thermoplastic material using microalgae as raw material, as in the Examples, carbon fixation occurs due to photosynthesis of microalgae. As a result, in calculating the total carbon emission factor of the Examples, there is an effect of reducing the total carbon emission factor by applying a negative carbon emission factor of −2.57 kgCO₂ eq.

This demonstrates that the Examples significantly reduce carbon emissions compared to the Comparative Examples when producing the same thermoplastic material.

Additionally, it may be seen from Table 2 that in each plastic material, the Examples show a carbon reduction rate of 3.65% or more compared to the Comparative Examples. In particular, Thermoplastic material F shows a maximum carbon reduction rate of 66.11%, indicating that carbon emissions were significantly reduced when microalgae raw materials were used compared to when petroleum raw materials were used.

In other words, the thermoplastic material according to the present disclosure may include thermoplastic resin derived from microalgae, and the thermoplastic resin may include at least one selected from the group consisting of polycarbonate-based resin, rubber-modified vinyl copolymer resin, styrene-based resin, and polyolefin-based resin, thus maintaining physical properties equal to or superior to conventional fossil fuel-based plastic materials while exhibiting a significantly lower level of carbon footprint, thereby reducing environmental burden.

The method of manufacturing the thermoplastic material according to the present disclosure may include cultivating microalgae to fix intracellular carbon in the microalgae, extracting microalgal oil from the microalgae, forming bio-naphtha from the microalgal oil, and forming a thermoplastic resin from the bio-naphtha, and due to the carbon fixation step resulting from photosynthesis of microalgae, there is an effect of reducing carbon emissions in the plastic material manufacturing process.

Additionally, the display device according to the present disclosure may include a display panel configured to display an image, a chassis configured to cover at least a portion of side surfaces and a portion of a front surface of the display panel, and a rear cover configured to cover a rear surface of the display panel, wherein the chassis and the rear cover include a thermoplastic material, the thermoplastic material being formed from thermoplastic resin derived from microalgae, thereby providing an eco-friendly display device with reduced environmental burden.

While the embodiments of the present disclosure have been illustrated and described above, the present disclosure is not limited to the specific embodiments described above, and various modifications may be made by those skilled in the art to which the present disclosure pertains without departing from the gist claimed in the claims.

What is claimed is:

1. A thermoplastic material for a display device, comprising:
a thermoplastic resin obtained from bio-naphtha derived from microalgae,
wherein the thermoplastic resin comprises at least one selected from the group consisting of polycarbonate-based resin, rubber-modified vinyl copolymer resin, styrene-based resin, and polyolefin-based resin.

2. The thermoplastic material of claim 1,
wherein the thermoplastic resin is present in an amount of 10 to 100 parts by weight based on 100 parts by weight of the thermoplastic material.

3. The thermoplastic material of claim 1,
wherein the microalgae comprises at least one selected from the group consisting of Chlorella, Euglena, Schizochytrium, Nannochloropsis, Isochrysis, Haematococcus, Dunaliella, Crypthecodinium, Tetraselmis, Neochloropsis, Picochlorum, Botryococcus, Coccolithophores, and Cyanobacteria.

4. The thermoplastic material of claim 1,
wherein the polycarbonate-based resin comprises at least one selected from the group consisting of polycarbonate (PC), polycarbonate/acrylonitrile-butadiene-styrene (ABS) blend resin, polycarbonate/polyester blend resin, polycarbonate/polyolefin blend resin, and polycarbonate copolymer.

5. The thermoplastic material of claim 1,
wherein the rubber-modified vinyl copolymer resin comprises at least one selected from the group consisting of acrylonitrile-butadiene-styrene (ABS), methyl methacrylate-butadiene-styrene (MBS), acrylonitrile-styrene-acrylate (ASA), acrylonitrile-EPDM-styrene copolymer (AES), and acrylonitrile-acrylate-styrene (AAS).

6. The thermoplastic material of claim 1,
wherein the styrene-based resin comprises at least one selected from the group consisting of polystyrene (PS), high impact polystyrene (HIPS), styrene-acrylonitrile (SAN), styrene-maleic anhydride (SMA), styrene-butadiene (SB), and styrene block copolymer (SBC).

7. The thermoplastic material of claim 1,
wherein the polyolefin-based resin comprises at least one selected from the group consisting of polypropylene (PP), high density polyethylene (HDPE), low density polyethylene (LDPE), linear low density polyethylene (LLDPE), ethylene-propylene copolymer (EP), ethylene-butene copolymer (EB), and ethylene-based elastomer.

8. The thermoplastic material of claim 1,
wherein a notched Izod impact strength of a ⅛″ thick specimen measured according to ASTM D256 is 3 kgf-cm/cm or more.

9. The thermoplastic material of claim 1,
wherein a flame retardancy of a 1.2 mm thick specimen measured according to UL-94 standard is V-0 or higher.

10. A method for manufacturing a thermoplastic material for a display device, the method comprising:
cultivating microalgae to fix intracellular carbon in the microalgae;
extracting microalgal oil from the microalgae;
forming bio-naphtha from the microalgal oil; and
producing a thermoplastic resin from the bio-naphtha.

11. The method of claim 10,
wherein the intracellular carbon in the microalgae is fixed in an amount of 2.57 kgCO₂ eq or more based on 1 kg of microalgal oil.

12. The method of claim 10,
wherein the forming of bio-naphtha comprises performing at least one of a hydrotreating process or a co-processing process to form bio-naphtha having a carbon number of C5 to C12.

13. The method of claim 10,
wherein the thermoplastic resin comprises at least one selected from the group consisting of polycarbonate-based resin, rubber-modified vinyl copolymer resin, styrene-based resin, and polyolefin-based resin.

14. The method of claim 10,
wherein a carbon reduction rate represented by the following expression (1) is 3.65% or more:
{(petroleum-based carbon emission factor)−(microalgae-based carbon emission factor)}/(petroleum-based carbon emission factor)×100(%) Expression (1):
wherein in expression (1), the petroleum-based carbon emission factor is a carbon emission factor when manufacturing plastic material from petroleum raw materials, and the microalgae-based carbon emission factor is a carbon emission factor when manufacturing plastic material from microalgae raw materials.

15. A display device comprising:
a display panel configured to display an image;
a chassis configured to cover at least a portion of side surfaces and a portion of a front surface of the display panel; and
a rear cover covering a rear surface of the display panel,
wherein the chassis and the rear cover comprise a thermoplastic material, the thermoplastic material comprising a thermoplastic resin obtained from bio-naphtha derived from microalgae,
wherein the thermoplastic resin comprises at least one selected from the group consisting of polycarbonate-based resin, rubber-modified vinyl copolymer resin, styrene-based resin, and polyolefin-based resin.

16. The method of claim 10,
wherein the thermoplastic resin is present in the thermoplastic material in an amount of 10 to 100 parts by weight based on 100 parts by weight of the thermoplastic material.
